(19) United States
(12) Patent Application Publication
(10) Pub. No.: US 2012/0129258 A1
(43) Pub. Date: May 24, 2012

(54) CELL CHIP AND METHOD OF FABRICATING THE SAME

(75) Inventors: Nam Seob BAEK, Daejeon (KR); Yong Hee Kim, Daejeon (KR); Ji Hyun Lee, Jeonju (KR); Sang Don Jung, Daejeon (KR)

(73) Assignee: Electronics and Telecommunications Research Institute, Daejeon (KR)

(21) Appl. No.: 13/289,266

(22) Filed: Nov. 4, 2011

(30) Foreign Application Priority Data
Nov. 23, 2010 (KR) 10-2010-0117091

(51) Int. Cl. C12N 5/071; B05D 3/00; B05D 5/00

(52) U.S. Cl. 435/402; 427/385.5

(57) ABSTRACT

Disclosed herein are a cell chip and a method of fabricating the same. The cell chip includes a cell-adhesivecell-adhesive layer disposed on a substrate. Photocrosslinked polymer barriers are disposed on the cell-adhesivecell-adhesive layer. The photocrosslinked polymer barriers may serve to restrict and grow cells only on the cell-adhesivecell-adhesive layer exposed between the barriers. Therefore, a cell growth direction may be precisely controlled. In addition, the photocrosslinked polymer barrier has a pattern formed by light, and simplifies a process of fabricating a cell chip.

### CROSS-REFERENCES TO RELATED APPLICATION

This patent application claims the benefit of priority from Korean Patent Application No. 10-2010-0117091 filed on Nov. 23, 2010 in the Korean Intellectual Property Office (KIPO), the entire contents of which are incorporated herein by reference.

### BACKGROUND OF THE INVENTION

1. Field of the Invention

The present invention relates to a cell chip and a fabricating method thereof.

2. Description of the Related Art

Bio chips are typically classified into DNA chips, protein chips and cell chips. While the DNA chips or protein chips are means for analyzing materials with using DNA or protein ligands on a substrate, the cell chips are means for analyzing materials through real-time reactions of living cells. The cell chips have advantages for detecting complex physiological signals of cells, which could not be measured by conventional methods.

One of the most notable fields of such cell chips is a neuron-on-a-chip using neurons. Generally, in the neurons, chemical stimulus signals detected at dendrites are converted into electrical signals at axons, and transferred to axon terminals. The electrical signals transferred to the axon terminals are converted into chemical stimulus signals, which are referred as neurotransmitters, and transferred across synapses as chemical stimulus signals to dendrites of different neurons. The neuron-on-a-chip may analyze the electrical signals using microelectrodes. In addition, the neuron-on-a-chip may serve as memories through formation of a neural network in which neurons are grown in a network form.

### SUMMARY OF THE INVENTION

In a cell chip, in order to modulate a direction of cell growth, a pattern is formed with a cell-adhesive material (CAM), such as poly-D-lysine, on a substrate by using a polydimethylsiloxane (PDMS) stamp. However, in this case, a neuron may be grown to a region in which a pattern is not formed with the cell-adhesive material, and therefore, confusion may be caused in a detected neuronal signal.

The present invention is provided in order to substantially obviate one or more problems due to limitations and disadvantages of the related art.

One object of present invention is to provide a cell chip that stably incubates cells and precisely controls a direction of cell growth and a method for fabricating the same.

The object of the present invention is not limited to the above-mentioned technical object, and other objects which are not mentioned will be clearly understood by the ordinary skilled in the art from the following description.

In order to achieve the objects, one aspect of the present invention provides one example of a cell chip. The cell chip includes a cell-adhesive layer disposed on a substrate. Photocrosslinked polymer barriers are disposed onto the cell-adhesive layer. The photocrosslinked polymer barriers may have biocompatibility. The photocrosslinked barriers may contain polyfluorene, in particular a funtionalized-polyfluorene.

Materials for forming the cell-adhesive layer may be synthetic polymers having amine groups in a main chain. The synthetic polymer may be polyethyleneimine.

The cell-adhesive layer may be a self-assembled monolayer. To this end, materials for forming the cell-adhesive layer may be synthetic polymers having amine groups in a main chain, and halosilyl, alkoxysilyl, thiol or disulfide groups in a side chain.

In order to achieve the object, one aspect of the present invention provides another example of a cell chip. Another cell chip includes a self-assembled polyethyleneimine layer on a substrate. Photocrosslinked polyfluorene barriers are disposed on the polyethyleneimine layer. The self-assembled polyethyleneimine layer may be formed of polyethyleneimine having halosilyl, alkoxysilyl, thiol or disulfide groups in a side chain. An electrode may be additionally disposed between the substrate and the polyethyleneimine layer.

In order to achieve the object, another aspect of the present invention provides a method for fabricating a cell chip. The method first includes forming a cell-adhesive layer on a substrate. A photocrosslinkable polymer layer is formed on the cell-adhesive layer. Photocrosslinked polymer barriers are formed by exposing and developing the photocrosslinkable polymer layer.

### BRIEF DESCRIPTION OF THE DRAWINGS

The above and other objects, features and other advantages of the present invention will be more clearly understood from the following detailed description taken in conjunction with the accompanying drawings, in which:

FIGS. 1A to 1E are schematic diagrams illustrating a method for fabricating a cell chip according to one example of the present invention;

FIG. 2 is a graph of a UV-vis absorption and fluorescence spectrum of a cell-repellent polymer (polyfluorene) layer before (dotted line) and after UV exposure (solid line) formed in an example of fabricating a cell chip;

FIG. 3A is an optical image of a photocrosslinkable polymer layer formed in an example of fabricating a cell chip, and FIG. 3B is an optical image of a photocrosslinked polymer pattern formed according to an example of fabricating a cell chip;

FIG. 4 is an optical image of cells incubated according to an example of incubating cells; and

FIG. 5A is an optical image of cells incubated according to an example of incubating cells, and FIGS. 5B to 5D are optical images taken after cells incubated according to an example of incubating cells are dyed.

### DESCRIPTION OF THE PREFERRED EMBODIMENTS

Example embodiments of the present invention are disclosed herein. However, specific structural and functional details disclosed herein are merely representative for purposes of describing example embodiments of the present invention, however, example embodiments of the present invention may be embodied in many alternate forms and should not be construed as limited to example embodiments of the present invention set forth herein.

Accordingly, while the invention is susceptible to various modifications and alternative forms, specific embodiments thereof are shown by way of example in the drawings and will herein be described in detail. It should be understood, however, that there is no intent to limit the invention to the particular forms disclosed, but on the contrary, the invention is to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present invention. As used herein, the term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (i.e., “between” versus “directly between,” “adjacent” versus “directly adjacent,” etc.).

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It should also be noted that in some alternative implementations, the functions/acts noted in the blocks may occur out of the order noted in the flowcharts. For example, two blocks shown in succession may in fact be executed substantially concurrently or the blocks may sometimes be executed in the reverse order, depending upon the functionality/acts involved.

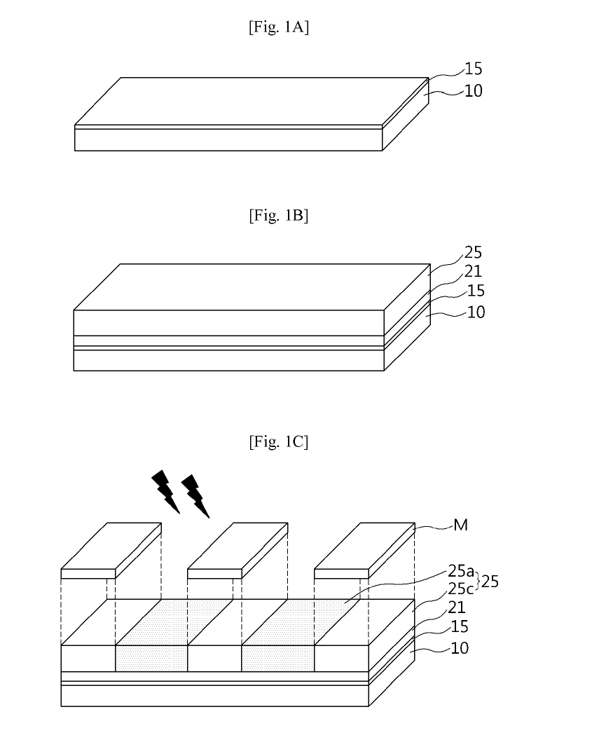

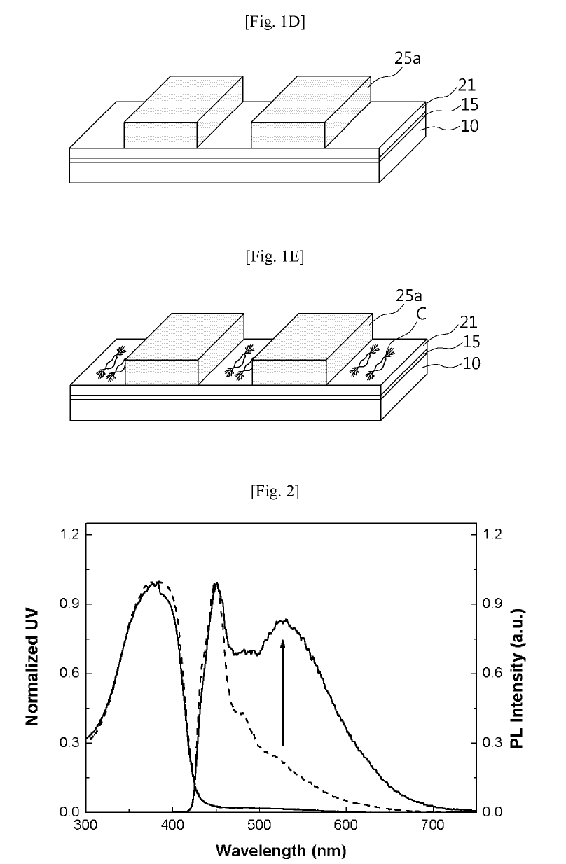

FIGS. 1A to 1E are schematic diagrams illustrating a method for fabricating a cell chip according to one example of the present invention.

Referring to FIG. 1A, a substrate 10 is provided. The substrate 10 may be a silicon substrate, a glass substrate, or a polymer substrate, for example, formed of polycarbonate, polystyrene or polyurethane. An electrode 15 may be formed on the substrate 10. The electrode 15 may be a metal oxide layer or a metal layer. The metal oxide layer may be an indium tin oxide (ITO) layer, an indium oxide (10) layer, a tin oxide (TO) layer, a fluorinated tin oxide (FTO) layer, an indium zinc oxide (IZO) layer or a zinc oxide (ZnO) layer, and the metal layer may be a Au layer, a Ag layer or a Pt layer. The electrode 15 may be patterned using a microarray, or may be omitted.

Referring to FIG. 1B, a cell-adhesive layer 21 is formed on the substrate 10. The cell-adhesive layer 21 may contain a synthetic polymer having an amine group in a main chain, for example, polyethyleneimine. Unlike a polypeptide such as polylysin, having an amide group which is easily hydrolyzable, the polyethyleneimine has a merit of a long lifespan. The amine group of the synthetic polymer may have a form of salt, and, therefore, a cation may be introduced into the synthetic polymer so as to easily and well immobilize a cell.

The cell-adhesive layer 21 may be a self-assembled monolayer (SAM) self-assembled on the electrode 15 (or on the substrate 10 when the electrode 15 is omitted). In this case, since the cell-adhesive layer 21 may bind to the electrode 15 (or the substrate 10 when the electrode 15 is omitted) by a chemical bond, the cell-adhesive layer 21 may firmly bind to the electrode 15 or substrate 10. To this end, a material for forming the cell-adhesive layer 21, for example, the synthetic polymer having the amine group, may have a halosilyl (e.g., chlorosilyl), alkoxysilyl (e.g., methoxysilyl or ethoxysilyl), thiol or disulfide group in a side chain. When the material for forming the cell-adhesive layer 21 has a halosilyl or alkoxysilyl group in the side chain, the cell-adhesive layer 21 may chemically bind to the electrode 15 or substrate 10 by a siloxane bond, thereby forming a self-assembled monolayer. When the material for forming the cell-adhesive layer 21 has a thiol or disulfide group in a side chain, the cell-adhesive layer 21 may chemically bind to the electrode 15 by a metal-sulfur bond, thereby forming a self-assembled monolayer.

Before the cell-adhesive layer 21 is formed on the electrode 21 or substrate 10, an OH group may be introduced to a surface of the electrode 15 or substrate 10.

A particular example of the material for forming the cell-adhesive layer 21 may be polyethyleneimine represented by the following Formula 1.

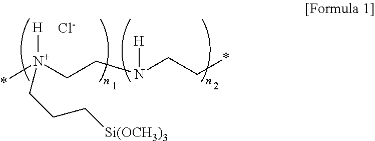

In the Formula 1, n₁ and n₂ are independent to each other, and are integers ranging from 1 to 20. Meanwhile, n_{z} may be 2 to 10 times, and preferably 4 times, larger than n₁.

A photocrosslinkable polymer layer 25 is formed on the cell-adhesive layer 21. The photocrosslinkable polymer layer 25 has no toxicity to cells, and thus has biocompatibility. The photocrosslinkable polymer layer 25 may contain a photocrosslinkable polymer, for example, a polyfluorene, preferably a functionalized-polyfluorene, more preferably the polyfluorene represented by the following Formula 2. The photocrosslinkable polymer contains a functional group (e.g., Br) generating a radical by light in a molecule. Therefore, the photocrosslinkable polymer layer 25 does not contain a photoinitiator which may have cytotoxicity. In addition, the photocrosslinkable polymer layer 25 may be a fluorescent polymer layer exhibiting fluorescence. A particular example of the material for forming the photocrosslinkable polymer layer 25 may be the polyfluorene represented by the following Formula 2.

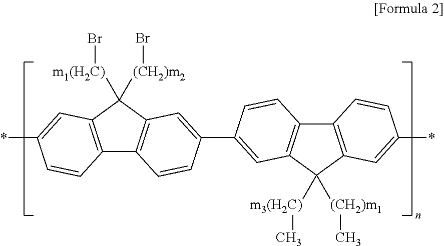

In Formula 2, m₁, m₂, m₃ and m₄ are independent to each other, and are integers ranging from 3 to 16. Moreover, n is an integer of 2 to 200.

Referring to FIG. 1C, a mask M is disposed on the photocrosslinkable polymer layer 25, and light is irradiated to the mask M. The light may be UV rays. In the photocrosslinkable polymer layer 25, regions to which light is irradiated 25a may be optically crosslinked, and regions 25c to which light is not irradiated may maintain an original state.

Referring to FIG. 1D, the photocrosslinkable polymer layer 25 to which light has been irradiated is developed to remove the regions 25c to which light is not irradiated. As a result, the regions 25a to which light is irradiated remain on the cell-adhesive layer 21, thereby forming photocrosslinked polymer barriers 25a. The cell-adhesive layer 21 is exposed between the barriers 25a. The barriers 25a and the cell-adhesive layer 21 exposed between the barriers 25a form a space for cell growth. Likewise, the barriers 25a are easily formed by light, and thus a method of fabricating a cell chip may become simple.

When the photocrosslinkable polymer layer 25 is a fluorescent polymer layer, the barriers 25a may exhibit fluorescence. In this case, the absence or presence of the barrier 25a may be identified more easily.

When the photocrosslinkable polymer layer 25 is a fluorescent polymer layer, the barriers 25a may exhibit fluorescence. In this case, the absence or presence of the barrier 25a may be identified more easily.

Referring to FIG. 1E, cells C are immobilized onto the cell-adhesive layer 21 exposed between the barriers 25a, and then incubated. The cells may be animal or plant cells or neurons. The cells may be restricted and grown only in the cell-adhesive layer 21 exposed between the barriers 25a, that is, a region defined by the barriers 25c. Thus, as the cell growth region is restricted by the barriers 25a, a cell growth direction may be precisely controlled. Meanwhile, since the barriers 25a are formed of a biocompatible polymer, cells in contact with the barriers 25a may not die, and thus a cell growth rate may be further increased.

Hereinafter, preferable examples are provided to help understanding of the present invention. However, the following examples are provided merely to help understanding of the present invention, not to limit the present invention.

### EXAMPLES

### 1. Fabricating Photocrosslinked Polymer

Monomers for synthesizing a polymer, 2,7-dibromo-9,9′-dihexylfluorene and 2,2′-(9,9-dioctyl-9H-fluorene-2,7-diyl)bis(4,4,5,5-tetramethyl-1,3,2-dioxaborolane), were synthesized with reference to the following articles [reference articles: (a) Zhou, Xing-Hua; Yan, Ji-Chao; Pei, Jian Macromolecules, 2004, 37, 7078-7080. (b) Hung, Ming-Chin; Liao, Jin-Long; Chen, Show-An; Chen, Su-Hua; Su, An-Chung J. Am. Chem. Soc. 2005, 127, 14576-14577. (c) Liu, Bin; Bazan, Guillermo C. J. Am. Chem. Soc. 2006, 128, 1188-1196, which are entirely incorporated herein as reference].

2,7-dibromo-9,9′-dihexylfluorene (yield: 70%):

¹H-NMR (CDCl₃, 300 MHz, ppm) δ 7.54-7.43 (6H, m, Ar—H), 3.32-3.27 (4H, t, J=6.9 Hz, CH₂), 1.95-1.90 (4H, m, CH₂), 1.72-1.62 (4H, m, CH₂), 1.25-1.03 (8H, m, CH₂), 0.64-0.53 (4H, m, CH₂)

2,2′-(9,9-dioctyl-9H-fluorene-2,7-diyl)bis(4,4,5,5-tetramethyl-1,3,2-dioxaborolane) (yield: 57%):

¹H-NMR (CDCl₃, 300 MHz, ppm) δ 7.8 (d, 2H), 7.74 (s, 2H), 7.72 (d, 2H), 1.9 (m, 4H), 1.4 (s, 24H), 1.0-1.2 (m, 20H), 0.8 (t, 6H), 0.5 (m, 4H)

As shown in the following Scheme, the synthesized 2,7-dibromo-9,9′-dihexylfluorene and 2,2′-(9,9-dioctyl-9H-fluorene-2,7-diyl)bis(4,4,5,5-tetramethyl-1,3,2-dioxaborolane) were added to a solution including toluene and tetrabutylammonium hydroxide (20 wt %) with 3 mol % Pd(PPh₃)₄ catalyst, and stirred at reflux, thereby synthesizing a photocrosslinkable polymer (A). After the reaction was completed, impurities were removed using a 0.2-μm filter and the resulting product was precipitated with a methanol solution, thereby obtaining a yellowish solid (yield: 67%, molecular weight (Mn)=1.52×10⁴ g/mol, PDI=2.3).

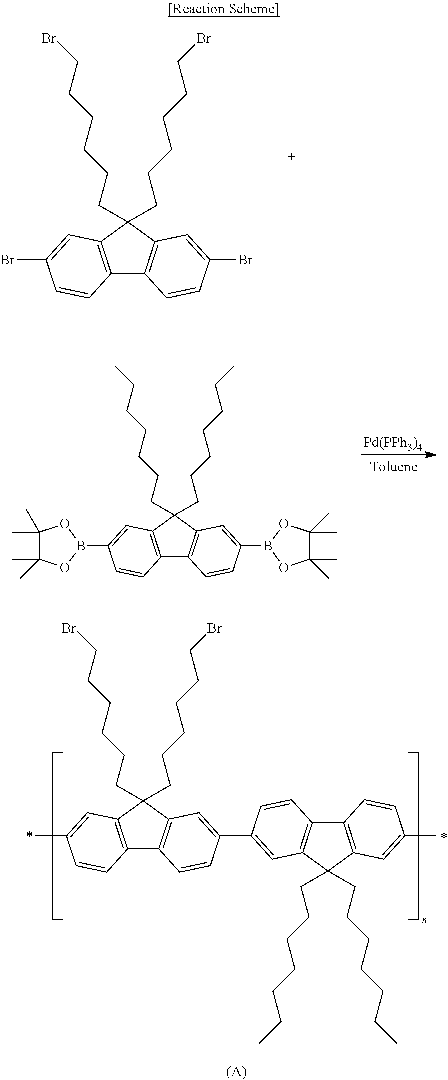

### 2. Fabricating Cell Chip

After an ITO substrate was cut in a size of 1 cm×1 cm, the substrate was put into a NH₃:H₂O₂:H₂O (1:4:20 v/v) mixed solution and treated at 70° C. for 1 hour, thereby forming an OH functional group on a surface of the ITO substrate. A modified-PEI solution (Gelest Co., 50 vol % in isopropanol) was diluted to 2 wt % using ethanol, and then treated on the ITO substrate having the OH functional group on the surface thereof. The modified-PEI solution and the ITO substrate were reacted at room temperature for 12 hours so as to chemically bind the denatured PEI to the ITO substrate. The PEI-binding ITO substrate was washed with ethanol, and thermally treated at 120° C. for 20 minutes. Afterward, the PEI-binding ITO substrate was washed for 5 minutes using an ultrasonic wave, washed again with ethanol and then dried with nitrogen, thereby removing a denatured PEI which did not participate in the chemical binding. A 1 wt % photocrosslinkable polymer (a) in chloroform (CHCl₃) solution was spin-coated on the ITO substrate having PEI chemically binding to a surface thereof at 1500 rpm for 30 seconds and then thermally treated at 70° C. for 1 minute, thereby forming a photocrosslinkable polymer layer. An optical mask was put on the photocrosslinkable polymer layer, and exposed using a contact aligner (MIDAS, Korea, P₃₆₅ₙₘ=45 mW) for 10 minutes. The exposed substrate was developed with an organic solvent (chloroform or THF) and dried with nitrogen, thereby forming a photocrosslinked polymer pattern.

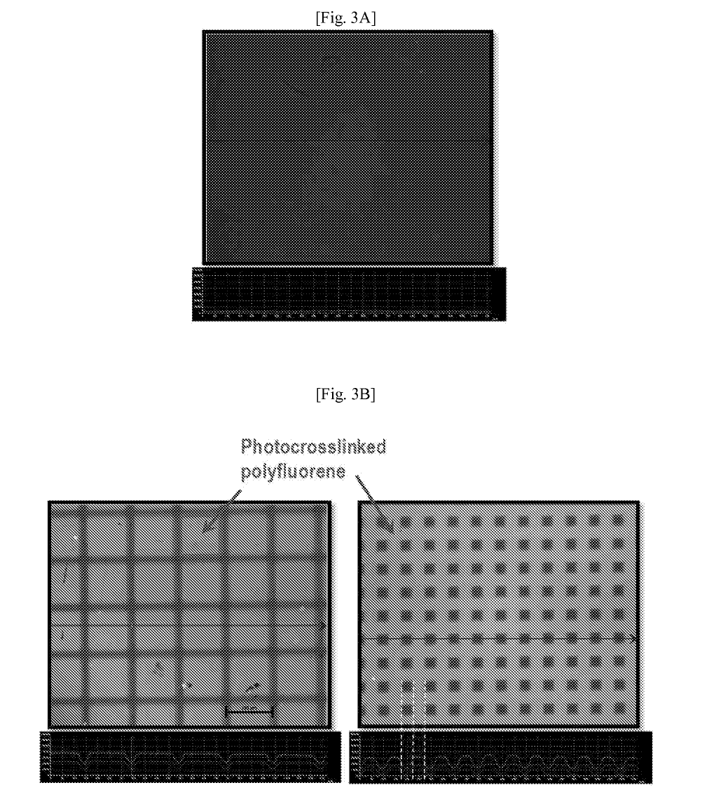

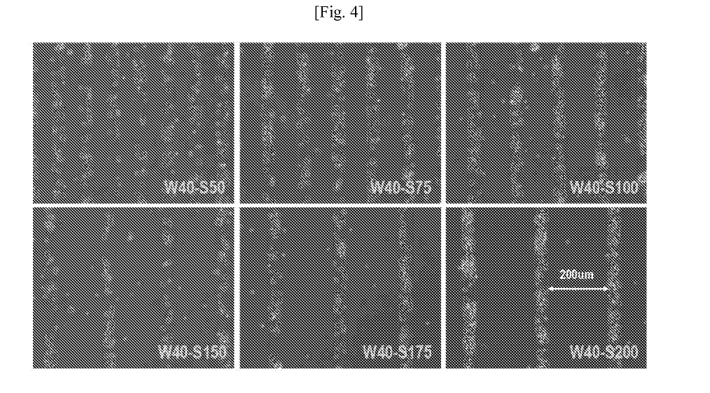

FIG. 2 is a graph of a UV-vis absorption and fluorescence spectrum of a uncrosslinked polymer layer (dotted line) and a photocrosslinked polymer pattern (solid line) formed in an example of fabricating a cell chip.

Referring to FIG. 2, it is seen that the uncrosslinked polymer layer (dotted line) and the photocrosslinked polymer pattern (solid line) absorbed light with a wavelength of approximately 380 nm, and emitted light with a wavelength of approximately 450 nm. In addition, it is known that the photocrosslinked polymer pattern (solid line) also emitted light with a wavelength of approximately 530 nm, which is caused by the crosslinking of the polymer.

FIG. 3A is an optical image of the photocrosslinkable polymer layer before UV exposure formed in an example of fabricating a cell chip, and FIG. 3B is an optical image of a photocrosslinked polymer pattern formed according to an example of fabricating a cell chip.

Referring to FIGS. 3A and 3B, it is seen that when UV rays were exposed to a photocrosslinkable polymer layer without a pattern (3A), a photocrosslinkable polymer layer (3B) with an island pattern and a line pattern was appropriately formed without a defect.

### 3. Incubating Cells

A pregnant mouse (ICR series) of 15-day was anesthetized with halothane and sacrificed by cervical dislocation. Subsequently, the body was washed with 70% ethanol and the fetus was extracted with the uterus from the abdominal region. After the separation of the fetus from the uterus, the scalp was dissected to extract brain tissue, and the brain tissue was put into a Hank's balanced saline solution (HBSS) in which there was neither Ca²⁺ nor Mg²⁺. After right and left cerebral cortices were separated, they were put into a 0.25% trypsin-containing HBSS, and the resulting solution was pipetted and then passed through a 10 ml syringe (needle size: 30 gauge) to separate cells. The separated cells were put into a 1 to 2 ml culture solution prepared by mixing 2 mM glutamine, B27 supplement, N₂ supplement and a neuronal growth factor with a Neurobasal (NB) media, and pipetted with a tapered pipette tip. Afterward, the number of cells was counted, and the cells were plated on a 12-well plate at a density of 7.0×10⁶/well. Then, the plate was incubated in a CO₂ incubator maintained under the conditions of 37° C., 5% CO₂ and 100% humidity.

Afterward, incubated cells were washed with DPBS (pH=7.4, phosphate buffered solution, 0.1 g/L CaCl₂, 0.2 g/L KCl, 0.2 g/L KH₂PO₄, 0.1 g/L/MgCl₂-6H₂O, 8.0 g/L NaCl, 2.16 g/L Na₂HPO₄-7H₂O) three times for 3 minutes, and fixed on the substrate prepared in <1. Fabricating a cell chip> using a fixative (% paraformaldehyde in PBS pH 7.4) at room temperature for 10 minutes. Then, the cells were washed with PBS three times for 3 minutes, and treated with a permeabilization solution (0.1% Triton X-100 PBS) at room temperature for 10 minutes to increase permeability of an antibody into the cell. After the cells were washed with PBS three times for 5 minutes, they were reacted with a blocking solution (2 wt % BSA in 0.1% tween20 PBS) at room temperature for approximately 30 hours to remove non-specific bindings of antibodies. After blocking, a primary antibody [Neuronal class III β-tubulin (TUJ1) monoclonal antibody (1:250 v/v)] was added, and a reaction was performed at 37° C. for 2 hours. After the reaction, the cells were washed with 0.1% Tween 20 PBS solution three times at room temperature each for 5 minutes, a secondary antibody [Alexa Fluor 488-conjugated goat anti-mouse IgG (1:500 v/v)] was added, and a reaction was performed at 37° C. for 30 minutes. After the reaction, the cells were washed with 0.1% Tween 20 PBS solution three times for 5 minutes, treated with DAPI (final concentration: 1 μg/ml) at room temperature for 10 minutes, and washed with 0.1% Tween 20 PBS solution three times for 5 minutes. Afterward, the cells were washed once with tertiary distilled water to remove a salt component from the 0.1% Tween 20 PBS solution, and observed using an Eclipse E600 to which a fluorescence observation apparatus was attached (Nikon).

FIG. 4 is an optical image of cells incubated according to Example of incubating cells.

Referring to FIG. 4, it is seen that cells were grown in a region defined by photocrosslinked polymer barriers.

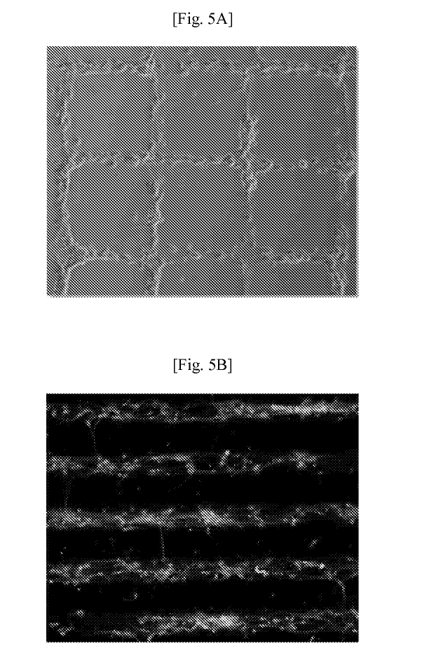

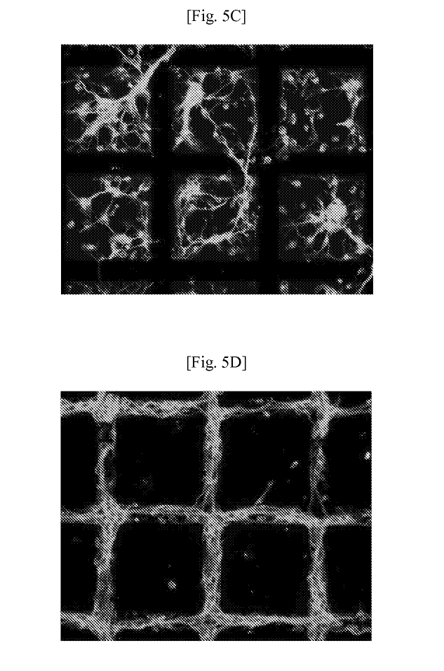

FIG. 5A is an optical image of cells incubated according to Example of incubating cells, and FIGS. 5B to 5D are optical images taken after cells incubated according to Example of incubating cells were dyed.

Referring to FIGS. 5A to 5D, it is identified that the incubated cells were relatively well grown in the region defined by the photocrosslinked polymer barriers.

According to the present invention described above, a cell chip has a cell-adhesive layer and photocrosslinked polymer barriers disposed on the cell-adhesive layer. The photocrosslinked polymer barriers can serve to restrict and grow cells only on the cell-adhesive layer exposed between the barriers. Therefore, a cell growth direction can be precisely controlled. In addition, the photocrosslinked polymer barrier has a pattern formed by light, and simplifies a process of fabricating a cell chip.

When the photocrosslinked polymer barrier can have biocompatibility, death of cells in contact with the barrier can be prevented, and the cell growth rate can be controlled. The photocrosslinked polymer barrier can be polyfluorene. Since the polyfluorene is a fluorescent polymer, an error of forming a pattern of a barrier can be easily confirmed.

A material forming the cell-adhesive layer may be a synthetic polymer having an amine group in a main chain, for example, polyethyleneimine. Unlike a polypeptide such as polylysin having an easily hydrolyzable amide bond, the synthetic polymer having the amine group in the main chain has a merit of a long lifespan. In addition, the cell-adhesive layer may be a self-assembled monolayer. In this case, binding ability between the cell-adhesive layer and the substrate is improved, and thus the lifespan of the cell chip may be extended.

While the example embodiments of the present invention and their advantages have been described in detail, it should be understood that various changes, substitutions and alterations may be made herein without departing from the scope of the invention.

What is claimed is:

1. A cell chip comprising:
a cell-adhesive layer disposed on a substrate; and
photocrosslinked polymer barriers disposed on the cell-adhesive layer.

2. The cell chip of claim 1, wherein the photocrosslinked polymer barriers have biocompatibility.

3. The cell chip of claim 2, wherein the photocrosslinked polymer barriers contain a functionalized-polyfluorene.

4. The cell chip of claim 1, wherein the cell-adhesive layer contains a synthetic polymer having an amine group in a main chain.

5. The cell chip of claim 4, wherein the synthetic polymer is polyethyleneimine.

6. The cell chip of claim 1, wherein the cell-adhesive layer is a self-assembled monolayer.

7. The cell chip of claim 6, wherein the cell-adhesive layer is formed of a synthetic polymer having an amine group in a main chain and a halosilyl, alkoxysilyl, thiol or disulfide group in a side chain.

8. A cell chip comprising:
a polyethyleneimine layer self-assembled on a substrate; and
photocrosslinked polyfluorene barriers disposed on the polyethyleneimine layer.

9. The cell chip of claim 8, wherein the self-assembled polyethyleneimine layer is formed of polyethyleneimine having a halosilyl, alkoxysilyl, thiol or disulfide group in a side chain.

10. The cell chip of claim 8, further comprising an electrode disposed between the substrate and the polyethyleneimine layer.

11. A method for fabricating a cell chip, comprising:
forming a cell-adhesive layer on a substrate;
forming a photocrosslinkable polymer layer on the cell-adhesive layer; and
forming photocrosslinked polymer barriers by exposing and developing the photocrosslinkable polymer layer.

12. The method of claim 11, wherein the photocrosslinked polymer barriers have biocompatibility.

13. The method of claim 12, wherein the photocrosslinkable polymer layer contains a functionalized-polyfluorene.

14. The method of claim 11, wherein the cell-adhesive layer is formed using a synthetic polymer having an amine group in a main chain.

15. The method of claim 14, wherein the synthetic polymer is polyethyleneimine
16. The method of claim 11, wherein the cell-adhesive layer is formed using a synthetic polymer having an amine group in a main chain and a halosilyl, alkoxysilyl, thiol or disulfide group in a side chain.
